(19) United States
(12) Patent Application Publication
(10) Pub. No.: US 2026/0256665 A1
(43) Pub. Date: Sep. 3, 2026

(54) CALCIUM HYDROXYAPATITE PARTICLES AND USE THEREOF FOR JUVENATION

(71) Applicant: MERZ NORTH AMERICA, INC., Raleigh, NC (US)

(72) Inventors: Bartosch NOWAG, Wiesbaden (DE); Janina KOLB, Hammersbach (DE)

(21) Appl. No.: 18/877,668

(22) PCT Filed: Jun. 28, 2023
(86) PCT No.: PCT/US2023/069248

§ 371 Date: Dec. 20, 2024

(30) Foreign Application Priority Data
Jun. 28, 2022 (EP) 22181450.2

(51) Int. Cl. A61K 8/24; A61K 8/02; A61K 9/00; A61K 31/728; A61K 33/42; A61Q 19/08

(52) U.S. Cl. CPC A61K 8/24; A61K 8/0245; A61K 9/0019; A61K 31/728; A61K 33/42; A61Q 19/08

(57) ABSTRACT

The present invention relates to a method for restoring cell cycle and proliferative competence in resting cells and/or stimulating proliferation of cells, wherein said method comprises the step of contacting the cells with calcium hydroxyapatite particles having a mean particle diameter of from 1 to 500 μm, as determined by light scattering. Furthermore, the present invention refers to such calcium hydroxyapatite particles for use in a method for treating or preventing a pathologic condition associated with arrested cell cycle, decreased proliferative competence, decreased proliferation rate, or a combination of two or more thereof, and the use thereof as anti-aging agent.

The present invention relates to a method for restoring cell cycle and proliferative competence in resting cells and/or stimulating proliferation of cells, wherein said method comprises the step of contacting the cells with calcium hydroxyapatite particles having a mean particle diameter of from 1 to 500 μm, as determined by light scattering. Furthermore, the present invention refers to such calcium hydroxyapatite particles for use in a method for treating or preventing a pathologic condition associated with arrested cell cycle, decreased proliferative competence, decreased proliferation rate, or a combination of two or more thereof, and the use thereof as anti-aging.

Humans and non-human animals are faced with signs of aging in many tissues of the body. In particular, aging of the skin and soft tissue is immediately visible as wrinkles and less tight soft tissue, preferably connective tissue. In particular, the skin visibly ages due to intrinsic and extrinsic factors, while the dermis and/or the epidermis may be affected (Lee et al., Int. J. Mol. Sci. 2021, 22:12489). But also other tissues such as muscles or the inner organs are faced by aging such as, e.g., liver cells, hepatocytes (Ogrodnik et al., Nature Communications, 2017, 8:15691). This may hamper organ functionality.

It is known that a major hallmark of aging is the accumulation of senescent cells, which have entered a state of cell cycle arrest typically considered as being irreversible after experiencing endogenous and environmental stresses (Song et al., Adv. Sci., 2020, 7(23):2002611; Di Micco et al., Nat. Rev. Mol. Cell. Biol., 2021, 22(2):75-95). Cellular senescence is a stable cell cycle arrest that is elicited in response to different stresses (Herranz and Gil, J. Clin. Invest., 2018, 128(4):1238-1246; Watanabe et al., CancerSci., 2017, 108:563-569). Song et al. teach a number of inducers and promoters of senescence as well as senolytic and senomorphic agents are enlisted. It is taught that physiologically autonomous clearance of senescent cells is compromised and dysfunctional cells accumulate generating a pro-inflammatory microenvironment. Di Micco et al. further explains molecular mechanisms in cellular senescence. Senescence-associated secretory phenotype (SASP) and experimental models including epithelial cells, pre-adipocytes and fibroblasts are further described in Cuolo et al. (Biology, 2029, 9:485).

Kirkland and Tchkonia (J. Intern. Med., 2020, 288(5):518-536) and Robbins et al., (Annu. Rev. Pharmacol. Toxicol., 2021, 61:779-803) teach several senolytic drugs that are intended to clear senescent cells and senomorphic drugs that are intended to convert senescent cells (SAPS) into rather harmless physiologic cells. Such senolytic drugs described therein may be small-molecular weight drugs also used in cancer therapy such as dasatinib or navitoclax, or cardiac glycosides such as digoxin, which may cause severe side effects.

There is still an unmet need for restoring cell cycle and proliferative competence in resting cells and/or stimulating proliferation of cells by further means that preferably have less side effects and are feasibly obtainable.

Surprisingly, it was found that a calcium hydroxyapatite (CaHA, Ca₅(PO₄)₃(OH)) particles, which are known as dermal fillers and in the context of stimulating the generation of collagen (Berlin et al., Semin. Cutan., Med. Surg., 2006, 25:132-137), can also be purposefully used for restoring cell cycle and proliferative competence in resting cells and/or stimulating proliferation of cells.

An aspect of the present invention relates to a method for restoring cell cycle and proliferative competence and/or stimulating proliferation of cells, wherein said method comprises the step of contacting the resting cells with calcium hydroxyapatite particles having a mean particle diameter of from 1 to 500 μm, as determined by light scattering.

In other words, the present invention relates to calcium hydroxyapatite particles having a mean particle diameter of from 1 to 500 μm, as determined by light scattering, for use in a method for restoring cell cycle and proliferative competence and/or stimulating proliferation of cells, wherein said method comprises the step of contacting the cells (preferably resting cells) with the calcium hydroxyapatite particles.

As used throughout the present invention, a mean particle diameter may be a weight average mean particle diameter. As the particles are essentially homogeneously formed, the weight average mean particle diameter essentially equals to the volume average mean particle diameter. The weight average mean particle diameter may, for instance, be detected by laser diffraction measurements. For instance, it may be detectable by ISO 13320:2009. Alternatively or additionally, the particle size distribution may be determined by microscopy and/or sieving.

Restoring cell cycle and proliferative competence may be considered as an anti-aging, a rejuvenating and/or a prejuvenating effect. This effect may be achieved on the level of cells, tissues and/or organs. For instance, the calcium hydroxyapatite particles of the present invention may transform skin into skin with younger characteristics and appearance by rejuvenation and/or even prevent skin aging by prejuvenation. All resulting in an anti-aging effect. Thus, more cells are restored in cell cycle and are proliferative competent in comparison to an untreated control cell population subjected to the same conditions except that no calcium hydroxyapatite particles of the present invention and no composition of the present invention, respectively is present.

Restoring cell cycle and proliferative competence may be understood in the broadest sense as transforming a resting cell into a cell that is able to proliferate via cell division. A resting cell is such that is not in cell cycle any more (non-proliferative cell), in other words that not able to divide proliferatively. The dividing competence may be resumed by the method of the present invention. Cycling cells may also be affected and represent a variant in increasing cell activity and amongst others, benefits for the skin by providing more active cells (e.g., fibroblasts), more protein production (e.g., collagens).

Stimulating proliferation of cells may be understood in the broadest sense as increasing the fraction of dividing cells. Thus, more cells proliferate in comparison to an untreated control cell population subjected to the same conditions except that no calcium hydroxyapatite particles of the present invention and no composition of the present invention, respectively is present.

The method may be a cosmetic (i.e., non-therapeutic) method.

Generally, the present invention also relates to calcium hydroxyapatite particles having a mean particle diameter of from 1 to 500 μm, which have been sintered at a temperature in the range of from 910 to 1030° C.

As used herein, the terms “calcium hydroxyapatite”, “calcium hydroxylapatite” and “basic calcium orthophosphate”, “calcium hydroxyphosphate”, “calcium phosphate tribasic”, “hydroxyapatite”, “hydroxylapatite”, and “tribasic calcium phosphate” and the abbreviations “CaHA” and “HAp” should be interchangeably understood in the broadest sense as commonly understood in the art. Calcium hydroxyapatite may be expressed by the formulae Ca₅(PO₄)₃(OH) and Ca₅[OHI(PO₄)₃], respectively.

Calcium hydroxyapatite is known as a biodegradable and bioresorbable material that may be used for bone repair (cf. LeGeros, Clinical Materials, 1993, 14:65-88).

Calcium hydroxyapatite particles generally usable are commercially available such as in the product Radiesse (Merz Pharmaceuticals GmbH, Germany/Merz North America, USA). Calcium hydroxyapatite particles of the present invention may also be considered as microspheres.

In a preferred embodiment, the calcium hydroxyapatite particles have been sintered at a temperature in the range of from 900 to 1300° C., or of from 910 to 1230° C., or of from 910 to 995° C., or of from 920 to 995° C., or from 930 to 990° C., or from 940 to 985° C., or from 950 to 980° C., or from 960 to 975° C., or of from 1130 to 1230° C. The calcium hydroxyapatite particles have preferably not been subjected to temperatures above the sintering temperature

In a preferred embodiment, the calcium hydroxyapatite particles have been sintered at a temperature in the range of from 910 to 1030° C. In a preferred embodiment, the calcium hydroxyapatite particles have been sintered at a temperature in the range of from 910 to 995° C., or of from 920 to 995° C., or from 930 to 990° C., or from 940 to 985° C., or from 950 to 980° C., or from 960 to 975° C. In a preferred embodiment, the particles are not subjected to a temperature of more than 995° C., or of more than 985° C., or of more than 980° C., or of more than 975° C. In a preferred embodiment, the particles were sintered at a temperature of (approximately) 970° C. In a preferred embodiment, the particles are not subjected to a temperature of more than the maximal temperature of the sintering. The calcium hydroxyapatite particles have preferably not been subjected to temperatures above the sintering temperature.

In another embodiment, the calcium hydroxyapatite particles have been sintered at a temperature in the range of from 1100 to 1300° C., or of from 1120 to 1270° C., or of from 1130 to 1230° C., or of from 1150 to 1200° C., or of from 1165 to 1175° C., or at a temperature of (approximately) 1170° C. The calcium hydroxyapatite particles have preferably not been subjected to temperatures above the sintering temperature.

It will be understood that also a mixture of particles (e.g., sintered at different temperatures) may be used.

The calcium hydroxyapatite particles of the present invention may have a well-defined higher surface area, which may enable particularly good restoration of cell cycle and proliferative competence and/or stimulating proliferation of cells and, optionally concomitantly increase of the collagen synthesis of fibroblasts.

Preferably, in the context of the present invention, the calcium hydroxyapatite particles are suitable for being injected. In other words, the calcium hydroxyapatite particles preferably are injectable calcium hydroxyapatite particles. In a preferred embodiment, the calcium hydroxyapatite particles have a mean particle diameter, of from 5 to 500 μm, or of 1 to 150 μm, or of from 2 to 100 μm, or of from 5 to 80 μm, or of from 10 to 60 μm, or of from 15 to 50 μm, or of from 20 to 45 μm, or of from 25 to 45 μm, as determined by light scattering.

Preferably, at least 80% by weight of the total mass of calcium hydroxyapatite particles is represented by calcium hydroxyapatite particles falling in an above size range and/or at least 80% of calcium hydroxyapatite particles in number fall within the above size ranges.

The calcium hydroxyapatite particles may have any shape. These may be spherical, ellipsoid, crystalline, random (also: irregular), or a mixture of two or more thereof. In a preferred embodiment, the calcium hydroxyapatite particles are (essentially) spherical or (essentially) ellipsoid. In a preferred embodiment, the calcium hydroxyapatite particles are (essentially) spherical. As used herein, the term “spherical” may be understood in the broadest sense as being substantially globular or ball-shaped, respectively. This does not necessarily mean perfect spheres, but characterizes the particles as not having sharp or angular edges.

In a preferred embodiment, the calcium hydroxyapatite particles are (essentially) spherical having a D-ratio above 0.7. The calcium hydroxyapatite particles more preferably have a D-ratio above 0.8, in particular above 0.9. In this context, a D-ratio of 1.0 indicates perfect roundness. Thus, the extensions in all three spatial directions is preferably substantially the same. The D-ratio may be determined by any means. As used herein, it is typically determined by means of microscopic imaging (also: by microscopy). For this purpose, microscopic images of individual particles are taken. Software conducts the measurements.

In a preferred embodiment, the calcium hydroxyapatite particles of the calcium hydroxyapatite particles have porous surfaces. Accordingly, the surfaces of the calcium hydroxyapatite particles are preferably not smooth and not having a tiled appearance. Preferably, the surfaces of the calcium hydroxyapatite particles bear numerous cavities. Preferably, also the inner of the calcium hydroxyapatite particles bear numerous pores/cavities. Thus, the calcium hydroxyapatite particles are preferably porous. The pores may be of any dimension.

In a preferred embodiment, the surfaces of the calcium hydroxyapatite particles have pores of an average diameter between 10 and 500 nm at the surface, as determined by Hg-porosimetry. In a preferred embodiment, the surfaces of the calcium hydroxyapatite particles have pores of a diameter between 10 and 100 nm diameter at the surface, as determined by Hg-porosimetry. In a preferred embodiment, the surfaces of the calcium hydroxyapatite particles have pores of an average diameter between 10 and 400 nm at the surface as determined by Hg-porosimetry. Preferably, the surfaces of the calcium hydroxyapatite particles have pores of an average diameter between 20 and 300 nm, or between 30 and 250 nm, or between 50 and 220 nm, at the surface as determined by Hg-porosimetry.

Preferably, each calcium hydroxyapatite particle bears at least 10, or at least 100 of such pores at its surface. It will be understood that the presence of such pores does not exclude the optional presence of one or more pores having other dimensions. Microscopy usable for the determination of pores is preferably scanning electron microscopy (SEM).

Alternatively, the average diameter may be determined by microscopy.

The sintering time to which the calcium hydroxyapatite particles have been subjected may be adapted to the sintering temperature and/or the mean particle diameter. Preferably, the calcium hydroxyapatite particles have been sintered for several hours. Preferably, the calcium hydroxyapatite particles have been sintered until uniform solid particles are obtained. In a preferred embodiment, the calcium hydroxyapatite particles have been sintered for 1 to 24 hours. In a preferred embodiment, the calcium hydroxyapatite particles have been sintered for 2 to 12 hours, or 3 to 16 h In a preferred embodiment, the calcium hydroxyapatite particles have been sintered for 1 hour to 2 hours, 1 hour to 3 hours, 2 to 4 hours, 3 to 5 hours, 4 to 6 hours, 5 to 7 hours, 6 to 8 hours, 7 to 9 hours, 8 to 10 hours, 9 to 11 hours, 10 to 12 hours, 11 to 13 hours, 12 to 14 hours, 13 to 15 hours, 14 to 16 hours, or 12 to 24 hours.

In a preferred embodiment, the calcium hydroxyapatite particles are further characterized by two or more of the following features selected from the group consisting of:
(a) the calcium hydroxyapatite particles are spherical or ellipsoid, more preferably spherical, in particular spherical having a D-ratio above 0.7;
(b) the surfaces of the calcium hydroxyapatite particles have pores of an average diameter between 10 and 500 nm at the surface, as determined by Hg-porosimetry;
(c) the calcium hydroxyapatite particles have a mean particle diameter of from 5 to 500 μm, or of from 1 to 150 μm, or of from 2 to 100 μm, or of from 5 to 80 μm, or of from 10 to 60 μm, or of from 15 to 50 μm, or of from 20 to 45 μm, or of from 25 to 45 μm, as determined by light scattering;
(d) the calcium hydroxyapatite particles have been sintered at a temperature in the range of from 900 to 1300° C., or of from 910 to 995° C., or of from 920 to 995° C., or from 930 to 990° C., or from 940 to 985° C., or from 950 to 980° C., or from 960 to 975° C., or of from 1130 to 1230° C.; and
(e) the calcium hydroxyapatite particles have been sintered for 1 to 24 hours, 2 to 12 hours, or 3 to 16 h

In a preferred embodiment, the calcium hydroxyapatite particles have been sintered for 1 to 24 hours at a temperature of from 960 to 975° C. In a preferred embodiment, the calcium hydroxyapatite particles having a mean particle diameter in the range of from 25 to 45 μm, as determined by light scattering, have been sintered for 1 to 24 hours at a temperature of from 960 to 975° C.

In a preferred embodiment, the calcium hydroxyapatite particles having a mean particle diameter in the range of from 25 to 45 μm, as determined by light scattering, and having pores of a diameter of 10 to 500 nm on their surface, as determined by microscopy, have been sintered for 1 to 24 hours at a temperature of from 960 to 975° C.

In a preferred embodiment, the calcium hydroxyapatite particles have a mean particle diameter of from 10 to 60 μm, and have been sintered at a temperature in the range of 950 to 980° C. for 3 to 16 h

In a preferred embodiment, the calcium hydroxyapatite particles have a mean particle diameter of from 25 to 45 μm, and have been sintered at a temperature in the range of 960 to 975° C.

In a preferred embodiment, the calcium hydroxyapatite particles have a mean particle diameter of from 25 to 45 μm, a spherical shape having a D-ratio above 0.7, and have been sintered at a temperature in the range of 960 to 975° C.

In a preferred embodiment, the calcium hydroxyapatite particles have a mean particle diameter of from 25 to 45 μm, having pores of an average diameter between and 500 nm at the surface, as determined by Hg-porosimetry, and have been sintered at a temperature in the range of 960 to 975° C.

In a preferred embodiment, the calcium hydroxyapatite particles have a mean particle diameter of from 25 to 45 μm, having a spherical shape having a D-ratio above 0.7, having pores of an average diameter between 10 and 500 nm at the surface, as determined by Hg-porosimetry, and have been sintered at a temperature in the range of 960 to 975° C.

Optionally, the calcium hydroxyapatite particles may comprise one or more other metal ions besides calcium in the CaHA particle crystal structure, such as a metal ion selected from the group consisting of fluorine, sodium, lithium, potassium, silicon, magnesium, and a combination of two or more thereof.

The calcium hydroxyapatite particles may be prepared by any means known in the art. Suitable procedures are, for example, described in U.S. Pat. No. 6,537,574 and WO 2001/012247. In a preferred embodiment, a slurry of small-sized calcium hydroxyapatite grains/crystals may be spray-dried. Such slurry may have any content of calcium hydroxyapatite usable for the purpose of preparing calcium hydroxyapatite by spray-drying. In one embodiment, the content of calcium hydroxyapatite in the slurry is set to 5 to 80% by weight, to 10 to 60% by weight, or to 20 to 40% by weight. In a preferred embodiment, for this purpose, the slurry may be pumped through a nozzle to form spherical particles that may be led through a column of heated air to remove the moisture. The size of the particles may be set by the choice of the nozzle. The particle size may be further improved by sieving different fractions. The obtained un-sintered particles may be sintered at the desired temperature as defined herein until the sintering has baked the previous submicron grains/crystals into uniform solid particles. Thus, the grains/crystals typically fuse and, thereby, enhance hardness.

As used herein, the slurry of submicron grains/crystals of calcium hydroxyapatite particles usable for preparing the calcium hydroxyapatite particles may be prepared by any means. For instance, it may be prepared by elutriating optionally commercially available calcium hydroxyapatite powder of a submicron grain size in water or an aqueous buffer or an aqueous and/or organic solution. Alternatively or additionally, the grains/crystal or preferably submicron size may also be prepared.

This may be achieved by admixing one or more soluble solutions of soluble calcium salt (e.g., calcium nitrate, calcium chloride, etc.) and a one or more soluble solutions of soluble hydrogen phosphate or dihydrogen phosphate (e.g., diammonium hydrogen phosphate). The mixing may be performed under vigorous mixing in order to obtain small-sized grains/crystals. Optionally, the pH may be adjusted to basic pH. Then, a slurry may be directly obtained. Optionally, the slurry may also be aged for several hours. Optionally, the crystal may be washed by one or more centrifugation/washing steps. Optionally, the slurry may further comprise of one or more wetting agents and/or binders such as polysorbate, sodium oxalate, polyvinyl alcohol, dextrin and/or carbon wax may be added.

Optionally, the calcium hydroxyapatite particles may comprise in the inside and/or may be coated with one or more agents stimulating neocollagenesis such as, e.g., polypeptides and/or small-molecular weight compounds (i.e., compounds having a molecular weight of not more than 1000 Da, of not more than 750 Da, or of not more than 500 Da) stimulating neocollagenesis.

As indicated above, the calcium hydroxyapatite particles of the present invention may be administrable to a subject by means of injection, in particular to a subject's skin and/or soft tissue, preferably connective tissue.

In a preferred embodiment, the calcium hydroxyapatite particles form part of a composition, preferably an injectable composition, comprising (or consisting of):
(A) the calcium hydroxyapatite particles as component A;
(B) one or more cosmetically and/or pharmaceutically acceptable carriers as component B comprising at least one viscous or liquid carrier;
(C) optionally one or more hyaluronic acids as component C, preferably wherein the one or more hyaluronic acids are non-crosslinked and/or crosslinked;
(D) optionally one or more local anesthetics as component D; and
(E) optionally one or more cosmetically and/or pharmaceutically acceptable additives other than components A, B, C and D as component E.

Accordingly, a further aspect of the present invention relates to composition, preferably an injectable composition, comprising (or consisting of):
(A) the calcium hydroxyapatite particles as component A;
(B) one or more cosmetically and/or pharmaceutically acceptable carriers as component B comprising at least one viscous or liquid carrier;
(C) optionally one or more hyaluronic acids as component C, preferably wherein the one or more hyaluronic acids are non-crosslinked and/or crosslinked;
(D) optionally one or more local anesthetics as component D; and
(E) optionally one or more cosmetically and/or pharmaceutically acceptable additives other than components A, B, C and D as component E.

It will be understood that the definitions and preferred embodiments as laid out in the context of the method of the present invention mutatis mutandis apply to the composition.

As used herein, the terms “composition” and “formulation” may be understood in the broadest sense interchangeably.

As used herein, the terms “component” and “ingredient” may be understood interchangeably in the broadest sense as a part of the composition of the present invention.

As used herein, the term “pharmaceutically acceptable” may be understood in the broadest sense as any being reasonably usable in a pharmaceutical context. As used herein, the term “cosmetically acceptable” may be understood in the broadest sense as any being reasonably usable in a cosmetic context. It will be understood that a pharmaceutically acceptable component or composition will typically also be inherently usable as being cosmetically acceptable. A cosmetically and/or pharmaceutically acceptable component or composition bears a low toxicity and can be administered to a human or non-human animal (typically mammal) body without seriously harm this human or non-human animal.

Preferably, the composition of the present invention is a cosmetically and/or pharmaceutically acceptable composition. The composition of the present invention may have any galenic form. In one embodiment of the present invention, the composition of the present invention is an injectable composition that is liquid or viscous. In another embodiment, the composition of the present invention is an injectable composition that is pasty. In a preferred embodiment, such composition can be considered as a dermal filler.

In a preferred embodiment, the composition is injectable into the skin or into other soft tissue, preferably connective tissue. Preferably, the composition is injectable in skin or other soft tissue, preferably connective tissue, improvement. In a preferred embodiment, the composition is injectable (sub)cutaneously/(sub)dermally.

Preferably, the composition is suitable for injection into a mammal, in particular a human. Preferably, the composition of the present invention is preferably (essentially) sterile and is preferably a-pyrogenic.

As used herein, the terms “liquid”, “viscous” and “pasty” may be understood in accordance with general understanding in the art. Preferably, “liquid” as used in the context of the present invention means having a viscosity of less than 10 mPas (millipascal-seconds, at standard conditions, 20° C., at 1013.25 hPa).

Preferably, “viscous” as used in the context of the invention means having a viscosity of from 10 to 1000 mPas (at standard conditions, 20° C., at 1013.25 hPa). The terms “viscous”, “gel” and “gel-like” should be understood interchangeably. Preferably, “pasty” as used in the context of the present invention means having a viscosity of from 1000 to 1,000,000 mPas (at standard conditions, 20° C., at 1013.25 hPa). These viscosity values can be determined by any means, for example, by a rotational/oscillating viscometer, e.g., according DIN 53019-4:2016-10). According to the invention, when the composition is a liquid, viscous or pasty composition, the calcium hydroxyapatite particles (component A) are preferably dispersed in the composition, i.e., in the liquid, viscous or pasty component of the composition. Accordingly, a liquid, viscous or pasty composition is typically a dispersion.

The composition of the present invention comprises one or more pharmaceutically acceptable carriers as component B, wherein at least one carrier preferably is a pasty, viscous or liquid carrier.

A cosmetically and/or pharmaceutically acceptable carrier (component B) according the present invention may be any carrier that is cosmetically and/or pharmaceutically acceptable, therefore, any carrier that is (essentially) non-toxic to the human or non-human animal (typically mammal) body. The one or more pasty, viscous or liquid carriers may be any cosmetically and/or pharmaceutically acceptable carrier that is pasty, viscous or liquid. For instance, the one or more pasty, viscous or liquid carriers may optionally comprise one or more cosmetically and/or pharmaceutically acceptable solvents such as, e.g., glycerol, water, an aqueous buffer (e.g., a saline or phosphate buffered saline), dimethyl sulfoxide (DMSO), ethanol, vegetable oil, paraffin oil, or combinations thereof. In one embodiment of the present invention, the one or more pasty, viscous or liquid carriers may comprise or consist of an a pyrogenic isotonic buffer, such as a physiological saline solution or a buffered physiological saline solution.

In a preferred embodiment, the sum of all cosmetically and/or pharmaceutically acceptable carriers (component B) comprises at least 10% by weight, at least 20% by weight, at least 50% by weight, at least 60% by weight, at least 70% by weight, or at least 80% by weight, or at least 90% by weight, referred to component B, of one or more pasty, viscous or liquid carriers.

In one embodiment of the present invention, the cosmetically and/or pharmaceutically acceptable carrier (component B) comprises at least 50% by weight, at least 60% by weight, at least 70% by weight, or at least 80% by weight, or at least 90% by weight, referred to component B, of glycerol. The one or more types of calcium hydroxyapatite particles (component A) may be dispersed in this carrier.

In a preferred embodiment, the one or more cosmetically and/or pharmaceutically acceptable carriers (component B) are selected from the group consisting of one or more polysaccharide derivatives or cosmetically and/or pharmaceutically acceptable salts thereof, one or more polysaccharides or cosmetically and/or pharmaceutically acceptable salts thereof, glycerol, water, one or more aqueous buffers, and combinations or two or more thereof. In a preferred embodiment, the one or more cosmetically and/or pharmaceutically acceptable carriers (component B) comprise one or more liquid, viscous or pasty components such as, e.g., glycerol, water, one or more aqueous buffers, and combinations or two or more thereof. This may make the composition injectable.

The one or more polysaccharide or derivatives thereof or cosmetically and/or pharmaceutically acceptable salts may preferably have thickening properties. The one or more polysaccharide or derivatives thereof or cosmetically and/or pharmaceutically acceptable salts thereof may have any molecular weight. Preferably, their molecular weight is in the range of from 1 kDa to 10 MDa, more preferably from 5 kDa to 5 MDa. Also a mixture of polysaccharides or derivatives or salts thereof may be used. Such mixture may be of the same or different type of polysaccharides or derivatives or salts thereof and may have different molecular size. Polysaccharides or derivatives thereof or cosmetically and/or pharmaceutically acceptable salts thereof may be non-crosslinked or cross-linked.

As used herein, mean molecular weight may be determined by any routine means suitable for this purpose such as, e.g., gel permeation chromatography (GPC), size exclusion chromatography (SEC), measuring the thickening effect (viscosimetry), mass spectrometry, etc. The mean molecular masses of the soluble fraction of polysaccharides or derivatives thereof or cosmetically and/or pharmaceutically acceptable salts thereof are preferably determined by gel permeation chromatography (GPC). The mean molecular masses of the insoluble, gel-forming fraction of polysaccharides or derivatives thereof or pharmaceutically acceptable salts thereof are preferably determined by measuring the thickening effect (viscosimetry) by routine experiments (e.g., at 25° C. by an EP monograph method on an Ubbelohe viscometer). As used herein, 1000 kDa (kilodaltons) are 1 MDa (megadalton).

The polysaccharide or derivative or cosmetically and/or pharmaceutically acceptable salt thereof may optionally form a gel in combination with the one or more pasty, viscous or liquid cosmetically and/or pharmaceutically acceptable carriers. The polysaccharide or derivative or cosmetically and/or pharmaceutically acceptable salt thereof may optionally form a hydrogel in combination with the one or more pasty, viscous or liquid carriers. The polysaccharide or derivative or cosmetically and/or pharmaceutically acceptable salt thereof may optionally be partly or completely dissolved the one or more viscous or liquid carriers. The one or more types of calcium hydroxyapatite particles (component A) may be dispersed in the one or more pasty, viscous or liquid carriers.

In a preferred embodiment, component B comprises or consists of:
(B1) one or more liquid, viscous or pasty cosmetically and/or pharmaceutically acceptable carriers, in particular liquid, viscous or pasty cosmetically and/or pharmaceutically acceptable carriers selected from the group consisting of glycerol, water, one or more aqueous buffers, and combinations or two or more thereof; and
(B2) one or more solid cosmetically and/or pharmaceutically acceptable carriers, preferably one or more polysaccharides or derivatives thereof or cosmetically and/or pharmaceutically acceptable salts thereof, in particular polysaccharides or derivatives selected from the group consisting of cellulose derivative (e.g., carboxymethyl cellulose, carboxyethyl cellulose), cellulose, and mixtures of two or more thereof.

Component B may comprise components B1 and B2 in any content ratio. In a preferred embodiment, component B comprises or consists of:
0.1 to 99% by weight, or 50 to 99.9% by weight, or 75 to 99% by weight, referred to component B, of B1; and
0.1 to 99% by weight, or 0.1 to 50% by weight, or 1 to 25% by weight, referred to component B, of B2.

In a preferred embodiment, the one or more cosmetically and/or pharmaceutically acceptable carriers (component B) are selected from the group consisting of (one or more types of) carboxymethyl cellulose or cosmetically and/or pharmaceutically acceptable salts thereof, glycerol, water, one or more aqueous buffers, and combinations or two or more thereof.

In a preferred embodiment, the one or more cosmetically and/or pharmaceutically acceptable carriers (component B) comprise (or consists of) (one or more types of) carboxymethyl cellulose or cosmetically and/or pharmaceutically acceptable salts thereof and glycerol.

As noted above, the composition of the present invention may optionally comprise one or more hyaluronic acids as component C. Thus, in one embodiment of the present invention, the composition comprises one or more hyaluronic acids (component C).

As used herein, hyaluronic acid may be understood in the broadest sense as generally understood in the art. It may be an (essentially) non-sulfated glycosaminoglycan and its salts as generally understood in the art. In accordance with general understanding in the art, it will be understood that the term “hyaluronic acid” also includes its salts. Hyaluronic acid may, for instance, have the CAS numbers 9004-61-9, 31799-91-4 (potassium salt), or 9067-32-7 (sodium salt). The terms “hyaluronic acid”, “hyaluronan” and “hyaluronate” and the abbreviation “HA” as used herein may be understood interchangeably.

The one or more hyaluronic acids usable as component C may be as described in WO 2021/156345.

In an embodiment of the present invention, hyaluronic acid (component C) has a molecular weight in the range of from 1 kDa to 10 MDa, more preferably in the range of from 5 kDa to 5 MDa, or in the range of from 0.3 MDa to 5 MDa, or in the range of from 0.3 MDa to 1 MDa, or in the range of from 1 MDa to 5 MDa. Hyaluronic acids or cosmetically and/or pharmaceutically acceptable salts thereof may be non-crosslinked or cross-linked or may be a mixture of crosslinked and non-crosslinked. In an embodiment of the present invention, hyaluronic acid (component C) is non-crosslinked (i.e. not crosslinked). It will be understood that also a mixture of hyaluronic acids or cosmetically and/or pharmaceutically acceptable salts thereof may be used. Such mixture may have different molecular size. Hyaluronic acids or cosmetically and/or pharmaceutically acceptable salts thereof may be non-crosslinked or cross-linked or may be a mixture of crosslinked and non-crosslinked. In an embodiment of the present invention, hyaluronic acid (component C) is one or more types of high-molecular weight hyaluronic acid of a mean molecular weight in the range of from 1 to 5 MDa, or of from 1.2 to 3.5 MDa, or of from 1.4 to 3.2 MDa, or of from 1.6 to 3.0 MDa, or of from 1.8 to 2.8 MDa, or of from 2.0 to 2.6 MDa, or of from 2.0 to 2.6 MDa, or of from 2.2 to 2.4 MDa (component C1). In another embodiment of the present invention, hyaluronic acid (component C) is one or more types of low-molecular weight hyaluronic acid of a mean molecular weight in the range of from 5 to 100 kDa, or of from 5 to 80 kDa, or of from 5 to 60 kDa, or of from to 50 kDa, or of from 5 to 45 kDa, or of from 5 to 40 kDa (component C2).

In an embodiment of the present invention, hyaluronic acid (component B) comprises (or consists of):
(C1) one or more types of high-molecular weight hyaluronic acid of a mean molecular weight in the range of from 1 to 5 MDa as component C1, or
(C2) one or more types of low-molecular weight hyaluronic acid of a mean molecular weight in the range of from 5 to 100 kDa as component C2, or
(C3) a combination of components C1 and C2.

In a preferred embodiment, hyaluronic acid (component C) comprises C1 and C2 in a weight ratio of components C1: C2, referred to the dry matters of components C1 and C2, in the range of from 1000:1 to 1:1.

As noted above, the composition of the present invention may optionally comprise one or more local anesthetics as component D. Thus, in one embodiment of the present invention, the composition comprises one or more local anesthetics (component D). A local anesthetic may make injection into a subject more comfortable.

A local anesthetic (component D) may be any local anesthetic. Preferably, a local anesthetic (component D), if present, is selected from the group consisting of: lidocaine, ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and combinations of two or more thereof and salts thereof. Alternative local anesthetics and combinations and salts thereof may also be used as component D. Combinations of two or more of the mentioned anesthetic agents, for example a combination of lidocaine and other “caine”-anesthetics like prilocaine, may also be used herein. In a preferred embodiment, the local anesthetic (component D) is or comprises lidocaine

As noted above, the composition of the present invention may optionally comprise one or more cosmetically and/or pharmaceutically acceptable additives other than components A, B, C and D as component E. Thus, in one embodiment of the present invention, the composition comprises one or more cosmetically and/or pharmaceutically acceptable additives other than components A, B, C and D (component E).

Such cosmetically and/or pharmaceutically acceptable additive (component E) may be any further agent that is (essentially) non-toxic to the human or non-human animal (typically mammal) body. In a preferred embodiment, component E is a bioactive ingredient that has an impact on:
(a) restoring cell cycle,
(b) restoring proliferative competence of resting cells,
(c) anti-aging, in particular fostering juvenation of tissue (an anti-aging factor such as, e.g., a senotherapeutic agent),
(d) biostimulation such as on collagen production (neocollagenesis factor),
(e) further stimulating proliferation of cells (cell proliferation factor), or
(f) a combination of two or more thereof.

Such cosmetically and/or pharmaceutically acceptable additive (component E) may also be an ingredient not having any of the above activities.

For instance, component E may be or comprise a bioactive ingredient selected from the group consisting of one or more senotherapeutic agent such as, one or more senolytic agents and/or one or more senomorphic agents. A senolytic agent (also: senostatic agent) may be understood as an agent that specifically induces death of senescence-associated secretory phenotype (SASP) cells. A senomorphic agent may be understood as an agent that supresses phenotypes of senescence, including senescence-associated secretory phenotype (SASP) cells. Examples of such senotherapeutic agents are known in the art such as, e.g., from Song et al. (Adv. Sci., 2020, 7(23):2002611), Di Micco et al. (Nat. Rev. Mol. Cell. Biol., 2021, 22(2):75-95), Cuolo et al. (Biology, 2029, 9:485), Kirkland and Tchkonia (J. Intern. Med., 2020, 288(5):518-536), and Robbins et al., (Annu. Rev. Pharmacol. Toxicol., 2021, 61:779-803). It will be understood that such agents can be combined with the calcium hydroxyapatite particles of the present invention, in particular may be present as component E in a composition of the present invention.

For instance, component E may be or comprise one or more agents stimulating neocollagenesis such as, e.g., one or more polypeptides or cosmetically and/or pharmaceutically acceptable salts thereof, and one or more small-molecular weight compounds stimulating neocollagenesis or cosmetically and/or pharmaceutically acceptable salts thereof. Neocollagenesis may, for instance be stimulating the production of collagen, in particular collagen selected from collagen type III, collagen type I, or a combination of collagen type I and III. As used herein, in the context of a protein such as a collagen type, the term “production” may be understood in the broadest sense as generation of the protein such as one or more collagen types. This may be also understood as protein expression.

For instance, a cell proliferation factor may be transforming growth factor (TGF), such as TGF-beta.

For instance, a cosmetically and/or pharmaceutically acceptable additive (component E) may be an ingredient not having biostimulating activity. Such component E may exemplarily be selected from the group consisting of one or more detergents (e.g., sodium lauryl sulfate (SLS)/sodium doceyl sulfate (SDS)), one or more coloring agents (e.g., TiO₂, food coloring), one or more vitamins, one or more salts (e.g., sodium, potassium, magnesium, calcium, and/or zinc salts), one or more humectants (e.g., sorbitol, glycerol, mannitol, propylene glycol, polydextrose), one or more enzymes, one or more preserving agents (e.g., benzoic acid, methylparabene), one or more texturing agents (e.g., polyethylene glycol (PEG), sorbitol), one or more emulsifiers, one or more separating agents, one or more antioxidants, one or more herbal and plant extracts, one or more stabilizing agents, one or more polymers (e.g., hydroxypropyl methacrylamide (HPMA), polyethylene imine (PEI), polyethylene glycol (PEG)), one or more uptake mediators (e.g., polyethylene imine (PEI), dimethyl sulfoxide (DMSO), a cell-penetrating peptide (CPP), a protein transduction domain (PTD), an antimicrobial peptide, etc.) one or more antibody/antibodies, one or more counterstain dyes (e.g., fluorescein, fluorescein derivatives, Cy dyes, an Alexa Fluor dyes, S dyes, rhodamine, quantum dots, etc.), one or more cell proliferation factors, one or more homeopathic ingredients, and combinations of two or more thereof. A dye may either improve localization of the injection (e.g., a cosmetically and/or pharmaceutically acceptable fluorescent dye like fluorescein or rhodamine) or may improve invisibility of the otherwise whitish composition of the present invention (e.g., by rendering it flesh-colored).

The optional further components C, D and/or E may be partly or completely comprised in the liquid, viscous or pasty component of the composition or may be dispersed therein. In a preferred embodiment, the composition of the present invention is a gel. Thus, it is preferably a gel-like, i.e., pasty or viscous, composition.

The components A and B and optionally C and optionally D an optionally E may be comprised in the composition in any content ranges and ratios.

In a preferred embodiment, the composition comprises at least 1% by weight, or at least 5% by weight, or at least 10% by weight, or at least 20% by weight, or at least 30% by weight, or at least 40% by weight, or at least 50% by weight, referred to the composition, of one or more types of calcium hydroxyapatite particles as component A. In a preferred embodiment, the composition comprises 1 to 80% by weight, 5 to 90% by weight, 10 to 80% by weight, 20 to 77% by weight, 30 to 75% by weight, 40 to 73% by weight, 50 to 72% by weight, 50 to 80% by weight, or 55 to 70% by weight, referred to the composition, of one or more types of calcium hydroxyapatite particles as component A. The weight percentages related to component A refer to dry matter of component A.

In a preferred embodiment, the composition comprises up to 80% by weight, 1 to 80% by weight, 2 to 75% by weight, 3 to 70% by weight, 4 to 65% by weight, 5 to 60% by weight, 10 to 55% by weight, 20 to 50% by weight, or 30 to 50% by weight, referred to the composition, of one or more types of pharmaceutically acceptable carriers as component B.

In a preferred embodiment, the composition comprises up to 10% by weight, 0.001 to 10% by weight, 0.001 to 5% by weight, 0.01 to 3% by weight, or 0.1 to 2% by weight, referred to the composition, of one or more local anesthetics as component C. In case component C in pure form is a solid compound, the weight percentages related to component C may refer to dry matter of component C.

The optional content ranges of the composition comprising one or more hyaluronic acids (optional component C) may be described as in WO 2021/156345. In a preferred embodiment, the composition comprises at least 1% by weight, or at least 5% by weight, or at least 10% by weight, or at least 15% by weight, or at least 20% by weight, or at least 25% by weight, or at least 30% by weight, referred to the composition, of one or more hyaluronic acids as component C. In a preferred embodiment, the composition comprises 0 to 50% by weight, 0.1 to 50% by weight, 0.5 to 45% by weight, 1 to 30% by weight, 5 to 25% by weight, or 25 to 50% by weight, referred to the composition, of one or more hyaluronic acids as component C. The weight percentages related to component C refer to dry matter of component C. In one embodiment, the weight ratio of components A:C, referred to the dry matters of components A and C, may be as far as component C is present in the composition, in the range of from 1000:1 to 1:10, or of from 100:1 to 1:10, or of from 50:1 to 1:5, or of from 20:1 to 1:4, or of from 10:1 to 1:3, or of from 5:1 to 1:2, or of from 2:1 to 1:1.

In a preferred embodiment, the composition comprises up to 10% by weight, 0.001 to 10% by weight, 0.01 to 5% by weight, or 0.1 to 2% by weight, referred to the composition, of one or more pharmaceutically acceptable additives other than components A, B, C and D as component E. In case component E in pure form is a solid compound, the weight percentages related to component E may refer to dry matter of component E.

In a preferred embodiment, the composition is an injectable composition consisting of (or comprising):
(A) 1 to 80% by weight, referred to dry matter, referred to the injectable composition, of one or more types of the calcium hydroxyapatite particles as component A;
(B) 1 to 80% by weight, referred to the injectable composition, of one or more cosmetically and/or pharmaceutically acceptable carriers as component B comprising at least one pasty, viscous or liquid carrier;
(C) 0 to 50% by weight (i.e., optionally up to 50% by weight), referred to dry matter, referred to the injectable composition, of one or more hyaluronic acids as component C, preferably wherein the one or more hyaluronic acids are non-crosslinked and/or crosslinked;
(D) 0 to 10% by weight (i.e., optionally up to 10% by weight), referred to the injectable composition, of one or more local anesthetics as component D; and
(E) 0 to 10% by weight (i.e., optionally up to 10% by weight), referred to the injectable composition, of one or more cosmetically and/or pharmaceutically acceptable additives other than components A, B, C and D as component E.

As used herein, a content by weight (e.g., % by weight) typically refers to the component as such. In case of a solid matter, it typically refers to the dry matter of the respective component (% by weight, referred to dry matter). It will be understood that all percentage numbers by weight of a composition sum up to 100% by weight. Thus, when the composition consists of components A, B, C, D and E, the sum of weight percentages of A, B, C, D and E is 100% by weight.

As used herein, the percentages by weight (% by weight) and the weight ratios of components are typically referred to the dry matters of the components. The terms “dry matter”, “dry weight” and “solid contend” may be understood interchangeably in the broadest sense as generally understood in the art. The person skilled in the art will notice that the dry matter may refer to the weight of the respective component without solvents/diluents and other components. The dry matters may also be considered when the respective components are dissolved, suspended or are forming a (hydro)gel with other components. In this case, the theoretical dry matter may be calculated, i.e., the weight of the solvents/diluents and further components may be subtracted from the total weight.

In a preferred embodiment, the composition is an injectable composition that comprises (or consists of):
(A) 1 to 80% by weight, referred to dry matter, referred to the injectable composition, of one or more types of calcium hydroxyapatite particles having a mean particle diameter of from 15 to 50 μm as component A;
(B) 1 to 80% by weight, referred to the injectable composition, of one or more pharmaceutically acceptable carriers as component B comprising or consisting of:
   (B1) 50 to 99.9% by weight of one or more liquid, viscous or pasty pharmaceutically acceptable carriers, in particular liquid, viscous or pasty pharmaceutically acceptable carriers selected from the group consisting of glycerol, water, one or more aqueous buffers, and combinations or two or more thereof; and
   (B2) 0.1 to 50% by weight of one or more solid pharmaceutically acceptable carriers, preferably one or more polysaccharides or derivatives thereof or pharmaceutically acceptable salts thereof, in particular polysaccharides or derivatives selected from the group consisting of cellulose derivative (e.g., carboxymethyl cellulose (CMC), carboxyethyl cellulose (CEC)), cellulose, and mixtures of two or more thereof;
(C) 0 to 50% by weight (i.e., optionally up to 50% by weight), referred to dry matter, referred to the injectable composition, of one or more hyaluronic acids as component C, preferably wherein the one or more hyaluronic acids are non-crosslinked and/or crosslinked;
(D) 0 to 3% by weight (i.e., optionally up to 3% by weight), referred to the injectable composition, of one or more local anesthetics as component D; and
(E) 0 to 10% by weight (i.e., optionally up to 10% by weight), referred to the injectable composition, of one or more pharmaceutically acceptable additives other than components A, B, C and D as component E.

Optionally, the composition of the present invention may be packaged. For instance, it may be packaged in syringes (for single use), vials, etc. A user manual may optionally be added to such package. Thus, the present invention also refers to a kit comprising the composition and a user manual for cosmetic and/or therapeutic uses of the present invention.

As indicated above, the calcium hydroxyapatite particles and/or the composition of the present invention may optionally be used for cosmetic (non-therapeutic) and therapeutic purposes. The calcium hydroxyapatite particles and/or the composition of the present invention is also usable as a soft tissue filler, in particular a dermal filler. Accordingly, the present invention also relates to the use of the calcium hydroxyapatite particles and/or the composition of the present invention for improving appearance of the skin and/or contour of a part of interest of the face or body of a subject. In particular, the present invention also relates to the use of the calcium hydroxyapatite particles and/or the injectable composition of the present invention as a soft tissue filler, in particular a dermal filler.

As indicated above, the calcium hydroxyapatite particles and/or the composition of the present invention are usable for restoring cell cycle and proliferative competence and/or stimulating proliferation of cells. The calcium hydroxyapatite particles and/or the composition of the present invention may be used to obtain an increased and prolonged collagen production (neocollagenesis). These results indicate that such composition is particularly well suitable for increasing expression of collagen. The calcium hydroxyapatite particles and/or the composition may bear particularly good biostimulation. This may lead to an increased skin quality, which may, for instance, include improvement of wrinkles, skin roughness, skin tightness and/or of signs of aging and facial contouring. It is, thus, a particularly suitable dermal and/or soft tissue filler.

By injecting the composition into certain areas/layer of a tissue, a very specific stimulation of the tissue is possible. By injecting the composition into certain areas/layer, a very specific stimulation of the skin is possible.

In a preferred embodiment, the cells contacted with the calcium hydroxyapatite particles are resting cells. The resting cells may be any cells that are resting, i.e., are typically not in cell cycle. In a preferred embodiment, the cells (preferably resting cells) are senescent cells.

The person skilled in the art understands the term “senescent cells” without burden. Cellular senescence is a stable cell cycle arrest that is elicited in response to different stresses (Herranz and Gil, J. Clin. Invest., 2018, 128(4):1238-1246; Watanabe et al., Cancer Sci., 2017, 108:563-569). It is considered as a growth arrest limiting the proliferative competence, i.e., the ability of cell division. Preferably and typically, senescent cells are stably arrested in either the G1 or G2/M phase of the cell cycle and are no longer able to divide, despite remaining viable and metabolically active for long periods (cf. Watanabe et al., Cancer Sci., 2017, 108:563-569). The phases of cell cycle are well known by those skilled in the art. In one embodiment, senescent cells are in G1 arrest and/or in G2/M arrest (cf. Gire and Dulic, Cell Cycle 14(3):297-304).

Cell cycle arrest in the G1 phase may result in non-cyclic cells and may be KI-67 negative. In contrast, cells in cell cycle may be KI-67 positive cells. KI-67 may be tested by any means. For instance, it may be detected by an anti-Ki67 antibody such as, e.g., the rabbit anti-Ki67 polyclonal antibody (affinity purified) commercially available as Abcam ab15580 (Abcam OLC, UK). A KI-67 staining shows whether a cell is in cell cycle and may also show whether a previously resting cell is subjected to restoring cell cycle and proliferative competence.

In the body, cellular senescence may decrease or even prevent unintended replication of old or damaged cells in that senescent cells exit cell cycle. Furthermore, senescent cells typically undergo further phenotypic alterations such as metabolic reprogramming, chromatin rearrangement, and/or autophagy modulation. In addition, senescent cells typically produce and secrete a complex combination of factors, collectively referred as the senescence-associated secretory phenotype (SASP). Such SASP is well-known in the art and is, for instance, described in Herranz and Gil (J. Clin. Invest., 2018, 128(4):1238-1246). Senescence and apoptosis are alternative cell fates of stressed cells.

As used herein, senescence may be any type of senescence. It may be age-related senescence. In one embodiment, senescence is replicative senescence, i.e., the exit of cell cycle and essential loss of proliferative competence of cells due to a plurality of cell divisions. Unlike germ cells and certain tissue stem cells, typical human somatic cells stop dividing permanently after a finite number of cell divisions in culture and enter a state of stable cell cycle arrest termed “cellular senescence” or “replicative senescence” (cf. Watanabe et al., Cancer Sci., 2017, 108:563-569). In another embodiment, senescence is contact-induced senescence caused by forming contact with adjacent cells and/or extracellular matrix (ECM). In another embodiment, senescence is senescence caused by a combination of replicative senescence and forming contact with adjacent cells and/or ECM. Senescence may also be caused by any other means.

The role of senescent cells is for instance known forfibroblasts in the context of skin aging. It is known that fibroblast senescence contributes to skin aging by secreting an SASP. As taught in Lee et al. (Int. J. Mol. Sci. 2021, 22:12489), an emerging hypothesis is that these changes, referred to as fibroblast senescence may be of interest in the context of skin aging due to their inherent ability to be almost free from apoptosis and to not be eliminated by the adaptive immune system. It is known that fibroblast senescence may contribute to skin aging by SASP. In this context, cell cycle and proliferation competence are typically decreased. Such decrease of cell cycle and proliferation competence, i.e., a high content of resting cells, may be caused by impairing the release of essential growth factors and enhances degradation of the extracellular matrix (ECM) such as, e.g., by activating matrix metalloproteinases (MMPs).

It will be understood by those skilled in the art that cellular senescence may be found in numerous different cell types. For instance, it is found in fibroblasts/fibrocytes (cf., Lee et al., Int. J. Mol. Sci. 2021, 22:12489) or in hepatocytes (Ogrodnik et al., Nature Communications, 2017, 8:15691). Thus, senescence cells may be found in any tissue, including inner and outer organs and tissues.

In a preferred embodiment, restoring cell cycle and proliferative competence is achieved for at least 0.1%, for at least 0.5%, for at least 1%, for at least 2%, for at least 5%, for at least 10%, or for at least 20%, of the cells (preferably previously resting cells) subjected to the calcium hydroxyapatite particles. In a preferred embodiment, restoring cell cycle and proliferative competence is increasing the fraction by at least 0.1%, by at least 0.5%, by at least 1%, by at least 2%, by at least 5%, for by least 10%, by at least 20%, by at least 30%, by at least 40%, bay at least 50%, by at least 75% or by 100% or more in comparison to a control cell population grow under the same conditions except the absence of the calcium hydroxyapatite particles of the present invention and the composition of the present invention, respectively. The resting cells may be any cells that are non-proliferative.

In a preferred embodiment, the cells (preferably resting cells) are selected from the group consisting of fibroblasts, fibrocytes, keratinocytes, melanocytes, adipocytes, and skin resident immune cells. In another embodiment, the cells (preferably resting cells) are another cell type such as, e.g., a hepatocyte. In a preferred embodiment, the cells (preferably resting cells) are selected from the group consisting of fibroblasts and fibrocytes, in particular are fibroblasts.

In a preferred embodiment, stimulating proliferation of cells is increasing the fraction by at least 0.1%, by at least 0.5%, by at least 1%, by at least 2%, by at least 5%, for by least 10%, by at least 20%, by at least 30%, by at least 40%, bay at least 50%, by at least 75% or by 100% or more in comparison to a control cell population grow under the same conditions except the absence of the calcium hydroxyapatite particles of the present invention and the composition of the present invention, respectively.

Restoring cell cycle and restoring proliferative competence of resting cells, in particular of senescent cells, may be accompanied by one or more further effects selected from the list consisting of increased epidermal and/or dermal thickness, improved keratosis scoring, reduced inflammation, increases in hair follicles density and/or numbering, increases in collagen types I and/or III, increases in elastin, increases in cells in cell cycle, decreases in skin inflammation, decreased matrix metalloproteases (MMPs), decreases in the level of reactive oxygen species (ROS), reduced rates of beta-galactosidase, decreased melanin content, and combinations of two or more thereof.

In a preferred embodiment, the cells (preferably resting cells) form part of a tissue. In a preferred embodiment, the cells (preferably resting cells) form part of a tissue in a living subject. In a preferred embodiment, the cells (preferably resting cells) form part of a tissue in a living subject and contacting the cells (preferably resting cells) with calcium hydroxyapatite particles is achieved by injecting an injectable composition of the invention into the tissue of interest.

The injection of the injectable composition into a tissue of interest may have an anti-aging, a rejuvenating and/or prejuvenating effect on that tissue.

As used herein, a rejuvenating effect may be understood in the broadest sense as an at least partial restoration of juvenile state. Thus, a cell or tissue that bears age-related phenotype may be at least partly juvenated.

As used herein, a prejuvenating effect may be understood in the broadest sense as an at least partial prevention of juvenile state (preferably of juvenile cells). Thus, a cell or tissue that does not bear age-related phenotype (e.g., is in cell cycle and proliferation competent) may be at least partly maintained in this state.

Anti-aging may be understood in the broadest sense and may be reversal of senescence. It may be used to terminate the inflammatory response of senescent cells and thereby aiming several age and inflammation associated diseases (e.g., rheumatoid arthritis), or atherosclerosis or type 2 diabetes).

The term “subject” (also: “individual” or “patient”) may be understood in the broadest sense as a human or non-human animal, typically a mammal, preferably a human or a domestic mammal, who/which can be subjected to a method of the present invention. As used herein, the term “mammal” may be understood in the broadest sense as any mammalian animal. Preferably, the mammal is a human or a domestic animal such as an animal selected from the group consisting of mouse, rat, cow, pig, dog, cat, horse. Particularly preferably, a subject as used herein is a human. A human or animal administered with the injectable composition of the present invention can also be designated as a patient, independent on his/her health state an irrespective whether clinical symptoms occur or do not occur.

Injecting into the skin of the part of interest of the face or body may be injection in any part of the skin. In one embodiment of the present invention, the composition of the present invention is administered to (in particular injected into) soft tissue. In one embodiment of the present invention, the composition of the present invention is administered to (in particular injected into) the dermis area, such as below the epidermis or above the hypodermis and as such may be injected subcutaneously/subdermally, hypodermically or intradermally, or some combinations. In one embodiment of the present invention, the composition of the present invention is administered (in particular injected) subcutaneously, subdermally, and/or intradermally. In a preferred embodiment, injecting into the skin of the part of interest of the face or body is injecting subcutaneously or intradermally. Injection may be performed by any means such as, e.g., by a syringe.

In another embodiment, the cells (preferably resting cells) form part of an in vitro cell culture. Then, the calcium hydroxyapatite particles may be admixed with the cell culture medium. The calcium hydroxyapatite particles may be used to rejuvenate senescent cells of interest. This may, for instance, be useful for research purposes, but also as a source of cells to be grafted (transplanted) to a host. Thus, in a preferred embodiment, the cells having restored cell cycle and proliferative competence are introduced into a tissue of interest. This tissue of interest may form part of an in vitro tissue culture/tissue model or may form part of a living subject. Such living subject may be subject of which the cells (preferably resting cells) were derived from or another subject (host of a xenographic transplantation of cells). The introduction of cells into a tissue of interest may have an anti-aging, a rejuvenating and/or prejuvenating effect on that tissue. In a preferred embodiment, the cells stimulated to proliferate are introduced into a tissue of interest.

Thus, in a preferred embodiment, the cells having restored cell cycle and proliferative competence and/or stimulated to proliferate are used for producing material of interest. Such material of interest may be obtained from cellular material as such (e.g., optionally dried lysate thereof or an isolated material obtained therefrom (e.g., a protein)), material secreted thereof (e.g., a protein of interest secreted thereby), or a combination of both.

The purpose of improving appearance of the skin and/or contour of a part of interest of the face or body of a subject may be understood in the broadest sense.

In a preferred embodiment, the method is further characterized in that it is a method for a purpose selected from the group consisting of reducing of wrinkles, improving facial lines, breast reconstruction or augmentation, rejuvenation of the skin, buttocks augmentation, remodeling of cheekbones, soft tissue augmentation, improving glabellar lines, improving nasolabial folds, improving marionette lines, improving buccal commissures, oral commissures, improving peri-lip wrinkles, improving crow's feet, improving subdermal support of the brows, malar and buccal fat pads, improving tear troughs, nose, augmentation of lips, augmentation of cheeks, augmentation of peroral region, augmentation of scars such as acne scars, augmentation of infraorbital region, resolving facial asymmetries, improving jawlines, augmentation of chin, restoring skin tightness, improving evenness of the skin, preventing or restoring discoloration of skin, improving skin glow, restoring skin hydration, improving skin barrier functions, and combinations of two or more thereof.

Overall skin quality may be improved. In a preferred embodiment, the method is a method for reducing of wrinkles (e.g. facial wrinkles), in particular fine wrinkles, of interest of the subject.

In a preferred embodiment, the method involves injecting the injectable composition subcutaneously or intradermally into the wrinkles of interest, preferably into the connective tissue of the subdermal skin.

In a preferred embodiment, the method of the present invention comprises the following steps:
(i) providing an injectable composition of the present invention; and
(ii) injecting said injectable composition into the skin of the part of interest of the face or body of a subject.

A further aspect of the present invention relates to the use of calcium hydroxyapatite particles having a mean particle diameter of from 1 to 500 μm, as determined by light scattering, as anti-aging agent restoring proliferative competence of resting cells, stimulating proliferation of cells and/or fostering juvenation of tissue.

It will be understood that the definitions and preferred embodiments as laid out in the context of the method of the present invention mutatis mutandis apply to the use. Preferably, the use may comprise contacting the calcium hydroxyapatite particles with the cells (preferably resting cells) of interest.

As used herein, the expression “juvenation of tissue” may be understood in the broadest sense as increasing the turnover of cells and/or reduction the content of senescent cells. It may comprise removal of senescent cells, replacement of senescent cells by non-senescent cells and rejuvenating senescent cells (i.e., restoring their proliferative competence).

As indicated above, the calcium hydroxyapatite particles and/or the composition of the present invention may also be used for therapeutic purposes. Accordingly, an aspect of the present invention relates to the calcium hydroxyapatite particles and/or the injectable composition of the present invention for use a medicament.

A further aspect of the present invention relates to calcium hydroxyapatite particles having a mean particle diameter of from 1 to 500 μm, as determined by light scattering, for use in a method for treating or preventing a pathologic condition associated with arrested cell cycle, decreased proliferative competence, decreased proliferation rate, or a combination of two or more thereof, in particular associated with undesired cellular senescence.

In other words, the present invention relates to a method for treating or preventing a pathologic condition associated with arrested cell cycle, decreased proliferative competence, decreased proliferation rate, or a combination of two or more thereof, in particular associated with undesired cellular senescence, wherein a sufficient amount of calcium hydroxyapatite particles having a mean particle diameter of from 1 to 500 μm, as determined by light scattering is administered to a subject in need thereof.

It will be understood that the definitions and preferred embodiments as laid out in the context of the method of the present invention mutatis mutandis apply to the calcium hydroxyapatite particles for use.

A pathologic condition may be understood in the broadest sense as any condition that deviates from a healthy state of a subject. A pathologic condition may be a disease (also disease illness, sickness, medicinal symptom, etc.). It may be or may not be associated with symptoms such as symptoms visible from the outside or measurable symptoms.

In a preferred embodiment, the pathologic condition is an age-associated pathologic condition. In a preferred embodiment, the pathologic condition is an age-associated skin pathologic condition.

An age-associated pathologic condition may be understood in the broadest sense as any pathologic condition that is associated with age/senescence of a subject of interest. The person skilled in the art is aware of such pathologic conditions. For example, an age-associated skin pathologic condition may be such as described in Blume-Peytavi et al., Gerontologist, 2016, 56(S2):S230-S242. For example, an age-associated skin pathologic condition may be selected from the group consisting of skin-related cancer (e.g., melanoma and other malignant neoplasms of skin (International Statistical Classification of Diseases and Related Health Problems (ICD) 10^{th} revision (ICD-10): 043), basal-cell carcinoma or squamous cell skin cancer (ICD-10: C44)), seborrheic dermatitis (ICD-10: L21), seborrhoea capitis (ICD-10: L21.0), actinic keratosis (ICD-10: L57.0), cutis laxa senilis (incl. elastosis) (ICD-10: L57.4), melanin hyperpigmentation (incl. lentigines) (ICD-10: L81.4), seborrheic keratosis (incl. dermatosis papulosa nigra) (ICD-10: L82), dry skin (incl. xerosis cutis, eczema craquele6) (ICD-10: L85.3)), pressure ulcer (ICD-10: L89), chronic wounds, increased risk for skin lesions and combinations of two or more thereof.

In a further embodiment, the pathologic condition is associated with pathologic deterioration of connective tissue, preferably wherein the pathologic condition is selected from the group consisting of urinary incontinence, vesicoureteral reflux, vocal cord augmentation, lipotrophy, in particular in a patient suffering from human immunodeficiency virus (HIV), a pathologic condition associated with age-related or pathologic deterioration of connective tissue, and combinations of two or more thereof.

In a further embodiment, the pathologic condition is selected from the group consisting of rheumatoid arthritis, atherosclerosis, Alzheimer's disease, amyotrophic lateral sclerosis, type 2 diabetes, and progeroid syndromes.

In a preferred embodiment, the pathologic condition is a pathologic skin condition. In a further embodiment, the pathologic condition is a non-skin pathologic skin condition. In a further embodiment, the pathologic condition is a condition on the connective tissue or liver. In a further embodiment, the pathologic condition in the brain or pancreas.

It is possible to support therapeutic treatment schemes. For instance, it is possible to support treatments related to tissue aging (e.g., skin aging) and/or senescent cells.

A further aspect of the present invention relates to the calcium hydroxyapatite particles of the present invention for use in a method of substituting or regenerating bone material (i.e., bones and bone grafts), implementing/fixing a tooth root, or filling a tooth. In other words, the present invention also relates to a method of substituting or regenerating bone material, implementing/fixing a tooth root, or filling a tooth in a subject, wherein said subject is administered with a sufficient amount of the calcium hydroxyapatite particles or the injectable composition of the present invention.

It will be understood that the definitions and preferred embodiments as laid out in the context of the method above mutatis mutandis apply to the injectable composition for use and to the methods of treating.

In a preferred embodiment, substituting or regenerating bone material includes the injection of the calcium hydroxyapatite particles of the present invention into or adjacent to the bone structure to be treated in the subject. Substituting or regenerating bone material can also be bone grafting. In a preferred embodiment, implementing/fixing a tooth root in includes the administration (e.g., injection) of the calcium hydroxyapatite particles of the present invention in the anchoring structure of the tooth. In a preferred embodiment, filling a tooth root in includes the administration of the calcium hydroxyapatite particles of the present invention in a cavity to be filled in eth tooth (e.g., a naturally occurring cavity of a drilled or milled cavity).

The Figures, Examples and claims described further illustrate the invention.

### EXAMPLES

### Example 1

### Preparation and Analysis of Calcium Hydroxyapatite (CaHA) Particles

### Preparation of a Slurry of Calcium Hydroxyapatite

### a.) Preparation of a Calcium Hydroxyapatite (CaHA) Slurry:

Calcium hydroxyapatite (CaHA) was precipitated via an aqueous slurry as described by Nieh et al. (Nieh, Choi and Jankowski, “Synthesis and characterization of porous hydroxyapatite and hydroxyapatite coatings”, Conference: 2001 Minerals, Metals& Materials Society Annual Meeting & Exhibition, New Orleans, LA (US), February 11-15, 2001). Thus, initially, a crystalline CaHA powder was prepared and precipitated by mixing calcium and phosphorous (e.g., Ca(OH)₂ and H₃PO₄) in a basic aqueous solution having a pH of approximately pH 11 (e.g., by NH₄OH) by mixing. CaHA crystals precipitate at room temperature. The precipitated CaHA slurry was purified by removal of excess reactants and byproducts using de-ionized water as described by Nieh et al. The purified CaHA slurry was concentrated via a decanting process and the CaHA crystals in the slurry were further reduced in size using a process such as a ball mill.

### b.) Alternative Preparation of a Slurry Based on Commercial Calcium Hydroxyapatite:

In the present invention, calcium hydroxyapatite powder of a submicron grain size is used. Such calcium hydroxyapatite powder of a submicron grain size is commercially available such as, e.g, from Millipore Sigma and Merck KGaA (Darmstadt, Germany). A slurry of the calcium hydroxyapatite powder is prepared by admixing the powder with water. The content of calcium hydroxyapatite in the slurry is set to 20 to 40% by weight.

### c.) Alternative Preparation of a Slurry Based on Generated Calcium Hydroxyapatite Nanocrystals:

3 parts by weight (wt.-parts) of calcium nitrate 4-hydrate are dissolved in approximately 44 wt.-parts of water. 1 wt.-part of diammonium hydrogen phosphate is dissolved in 31 wt.-parts of water. The obtained aqueous solution of diammonium hydrogen phosphate is added slowly to the aqueous solution of calcium nitrate under vigorous stirring. The pH of the obtained solution is adjusted to pH11 by means of sodium hydroxide. The slurry may be aged for several hours. Optionally, the crystal may be washed by one or more centrifugation/washing steps. Such procedure is described in Eslami et al., (Iranian Journal of Pharmaceutical Sciences, 2008, 4(2):127-134). The content of calcium hydroxyapatite in the slurry is set to 20 to 40% by weight.

Preparation and Sintering of Calcium Hydroxyapatite (CaHA) Particles from the Slurry

The CaHA slurry was formed into microspheres utilizing an atomizer/spray dryer as described by Nieh et al. Thus, the slurry is pressed through a nozzle into a warm space. Air classification or mechanical sieving was utilized to remove CaHA particles that are outside the desired diameter threshold. The remaining CaHA particles are sintered as described by Nieh et al. at a temperature of interest and time to control the crystalline structure/porosity of the particles. The sintered CaHA particles were granulated then washed/dried/sieved to achieve a powder consisting of singular CaHA particles of the desired size range.

The preparation of calcium hydroxyapatite (CaHA) particles may also be performed as described in U.S. Pat. No. 6,537,574 and WO 2001/012247.

### Analysis of Calcium Hydroxyapatite Particles

Calcium hydroxyapatite particles having a mean particle diameter of from 25 to 45 μm sintered at 970° C. and comparable particles sintered at 1170° C. were prepared. The size and shape distribution was analyzed by microscopic means and quantitatively by measuring light scattering.

It was visible that the size distribution is rather narrow. The vast majority of particles has a diameter of from 25 to 45 μm.

Scanning electron microscopy (SEM) images which were taken from three fractions per sample were used to determine the particle- and volume-weighted size distributions. Therefore, up to 400 SEM images per fraction were taken at a magnification of ×500 and further processed by the automated image analyzing software ImageJ (version 1.51j8). Image processing were provided for every fraction image after defined parameters like Feret diameter and aspect ratio (D-ratio). The averaged results finally represent particle- and volume-weighted size distributions based on at least more than 50,000 identified particles.

Results of a quantitative measurement are depicted in Table 1 below. No particles having a size of >125 μm were found.

TABLE 1
Quantitative comparison of calcium hydroxyapatite (CaHA)
particles having a mean particle diameter of 25 to 45
μm prepared at different sintering temperatures.

| | | | | |
|---|---|---|---|---|
| Sintering temperature of | 970° | C. | 1170° | C. |
| the CaHA particles | | | | |

| | | |
|---|---|---|
| Number of measures | 66,820 | 228,776 |

| | | | | |
|---|---|---|---|---|
| particles | | | | |
| Mean area of the outer | 538.9 | (±342.7) | 668.3 | (±271.4) |
| surface of the CaHA | | | | |
| particles in μm² | | | | |
| (±standard deviation) | | | | |
| Feret diameter in μm) | 27.6 | (±9.0) | 30.9 | (±6.6) |
| (±standard deviation) | | | | |
| Perimeter in μm | 93.5 | (±29.2) | 99.0 | (±21.3) |
| (±standard deviation) | | | | |
| D-ratio | 0.9 | (±0.1) | 0.9 | (±0.1) |

It was visible that both sintering temperatures led to particles of essentially spherical shape and well-defined size distribution. However, it was surprisingly found that pores (visible as dark spots) can be noted on the surface of the calcium hydroxyapatite particles sintered at 970° C., while the surfaces of the calcium hydroxyapatite particles sintered at 1170° C. were essentially smooth.

### Example 2

Restoration of cell cycle and proliferative competence in resting cells and stimulating proliferation of cells.

The CaHA particles as prepared according to Example 1 were tested in cell culture experiments. The cell culture experiment was conducted several times. The results depicted below represent the mean values of three independent experiments,

### a) Cultivation on a 96-Well Plate:

5000 normal human dermal fibroblasts (passage 5) were seeded per well. Cells were incubated with different amounts of microspheres. Two types of microspheres were used: First type has a rough surface (970° C.), the second type has a smooth surface (1170° C.). Concentration used for both types of microspheres were set to 1 mg/ml, 2 mg/ml, and 5 mg/ml.

For the analysis of cycling cells, 24 hours after seeding of cells, cells were incubated with different amounts of microspheres. 8 hours after incubation the cells were fixed and stained as described in the KI-67 staining protocol.

For the analysis of non-cycling cells (Confluence induced senescence), 8 days after seeding of cells, cells were incubated with different amounts of microspheres. 8 hours after incubation the cells were fixed and stained as described in the KI-67 staining protocol.

For the analysis of non-cycling cells (replication induced senescence), prior to exposing of cells to both microspheres, the cells were cultured for 8 weeks till their replication stops, non-replicative cells were incubated with different amounts of microspheres. 8 hours after incubation the cells were fixed and stained as described in the KI-67 staining protocol.

### b) Cultivation on a 48-Well Plate:

25000 normal human dermal fibroblasts (passage 5) were seeded per well. Cells were incubated with different amounts of microspheres. Two types of microspheres were used: First type has a rough surface; the second type has a smooth surface. Concentration used for both types of microspheres were 1 mg/ml, 2 mg/ml and 5 mg/ml. The cells were incubated with and without addition of 5 ng/ml of transforming growth factor beta (TGF).

For the analysis of cycling cells, 24 hours after seeding of cells, cells were incubated with different amounts of microspheres. 8 hours after incubation the cells were fixed and stained as described in the KI-67 staining protocol.

For the analysis of non-cycling cells (Confluence induced senescence), 8 days after seeding of cells, cells were incubated with different amounts of microspheres. 8 hours after incubation the cells were fixed and stained as described in the KI-67 staining protocol.

For the analysis of non-cycling cells (replication induced senescence), prior to exposing of cells to both microspheres, the cells were cultured for 8 weeks till their replication stops, non-replicative cells were incubated with different amounts of microspheres. 8 hours after incubation the cells were fixed and stained as described in the KI-67 staining protocol.

### c) KI-67 Staining Protocol:

After fixing the cells were blocked with a solution containing albumin, Triton X-100 and PBS (phosphate buffered saline) for 2 hours. KI-67 (Abcam ab15580) (a rabbit anti-Ki67 polyclonal antibody, affinity purified) (Abcam OLC, UK) was used in a concentration of 1 μg/ml over night at 4° C. Secondary antibody Alexa Fluor 488 was used for 1 hour at room temperature. To stain the cell membrane CellMask (Invitrogen, Thermo Fischer, USA) was used for 30 minutes at room temperature. DAPI (4′,6-diamidino-2-phenylindole) staining for 10 minutes was used to stain the nucleus of the cells. The cells were then measured using a cell analyzer InCell 2200 (GE Healthcare, USA). The quantification of KI-67 positive cells was done by a specific software InCarta (Molecular Devices, LLC, USA). In principle, cells were detected via the cell mask. The next step is the determination of the region of interest using the DAPI staining showing the nucleus of the cells. KI-67-positive cells appear green (Alexa Fluor 488) in the nucleus. The KI-67 negative and positive cells are mathematically divided and the ratio between those cells results in the percentage of cycling cells.

### d) Results

The results re-depicted in Tables 2, 3 and 4.

TABLE 2
Influence of CaHA particles on cycling cells (replicative fibroblasts,
cultivation for 24 hours before treatment) each compared to
the respective control (ctrl, without CaHA particles) with
and without transforming growth factor beta (TGF) (percentage
of cells in mean values three experiments):

| CaHA particles | Cells in cycle | % CTRL |
|---|---|---|
| ctrl. | 34.86 | 100.00 |
| 970° C. (1 mg/ml) | 53.12 | 152.37 |
| 970° C. (2 mg/ml) | 60.46 | 173.43 |
| 970° C. (5 mg/ml) | 63.42 | 181.91 |
| 1170° C. (1 mg/ml) | 33.01 | 94.69 |
| 1170° C. (2 mg/ml) | 26.97 | 77.37 |
| 1170° C. (5 mg/ml) | 33.64 | 96.50 |
| 5 ng/ml TGF | 41.32 | 100.00 |
| 970° C. (1 mg/ml) + TGF | 64.98 | 157.27 |
| 970° C. (2 mg/ml) + TGF | 68.86 | 166.65 |
| 970° C. (5 mg/ml) + TGF | 71.98 | 174.22 |
| 1170° C. (1 mg/ml) + TGF | 45.72 | 110.65 |
| 1170° C. (2 mg/ml) + TGF | 44.29 | 107.19 |
| 1170° C. (5 mg/ml) + TGF | 49.84 | 120.63 |

It was surprisingly found that contacting the cells with higher contents of CaHA particles of 5 mg significantly stimulated the proliferation of cells. In particular, the CaHA particles sintered at 970° C. showed a high dose-dependent stimulation efficacy. The growth factor TGF stimulates proliferation and its effect can be surprisingly further enhanced by the CaHA particles.

TABLE 3
Influence of CaHA particles on resting and, thus, non-cycling cells
(replicative fibroblasts, cultivation for 8 days before treatment)
each compared to the respective control (ctrl, without CaHA particles)
with and without transforming growth factor beta (TGF) (percentage
of cells in cycle, mean values of three experiments):

| CaHA particles | Cells in cycle | % CTRL |
|---|---|---|
| ctrl. | 2.14 | 100.00 |
| 970° C. (1 mg/ml) | 8.36 | 391.32 |
| 970° C. (2 mg/ml) | 16.39 | 767.02 |
| 970° C. (5 mg/ml) | 23.61 | 1105.25 |
| 1170° C. (1 mg/ml) | 3.48 | 162.93 |
| 1170° C. (2 mg/ml) | 4.07 | 190.44 |
| 1170° C. (5 mg/ml) | 6.32 | 296.00 |
| 5 ng/ml TGF | 4.55 | 100.00 |
| 970° C. (1 mg/ml) + TGF | 6.97 | 153.20 |
| 970° C. (2 mg/ml) + TGF | 11.69 | 256.88 |
| 970° C. (5 mg/ml) + TGF | 13.99 | 307.30 |
| 1170° C. (1 mg/ml) + TGF | 5.65 | 124.09 |
| 1170° C. (2 mg/ml) + TGF | 6.94 | 152.43 |
| 1170° C. (5 mg/ml) + TGF | 10.01 | 219.84 |

TABLE 4
Long-term influence of CaHA particles on resting and, thus, non-cycling
cells (senescent fibroblasts, cultivation for 8 weeks before treatment)
each compared to the respective control (ctrl, without CaHA particles)
with and without transforming growth factor beta (TGF) percentage
of cells in cycle, mean values of three experiments):

| CaHA particles | Cells in cycle | % CTRL |
|---|---|---|
| ctrl. | 6.16 | 100.00 |
| 970° C. (1 mg/ml) | 18.40 | 298.81 |
| 970° C. (2 mg/ml) | 36.01 | 584.85 |
| 970° C. (5 mg/ml) | 55.16 | 896.01 |
| 1170° C. (1 mg/ml) | 8.86 | 143.93 |
| 1170° C. (2 mg/ml) | 10.43 | 169.34 |
| 1170° C. (5 mg/ml) | 16.50 | 268.02 |
| 5 ng/ml TGF | 6.32 | 100.00 |
| 970° C. (1 mg/ml) + TGF | 19.81 | 313.56 |
| 970° C. (2 mg/ml) + TGF | 27.74 | 439.08 |
| 970° C. (5 mg/ml) + TGF | 39.00 | 617.43 |
| 1170° C. (1 mg/ml) + TGF | 9.00 | 142.42 |
| 1170° C. (2 mg/ml) + TGF | 9.32 | 147.55 |
| 1170° C. (5 mg/ml) + TGF | 12.35 | 195.52 |

It was surprisingly found that contacting the cells with CaHA particles enabled restoring cell cycle and proliferative competence in resting cells. While in control samples (crtl), virtually no cells were in cycle and all cells remained resting, in particular after 8 weeks, the addition of particles restored their cell cycle and proliferative competence by several orders of magnitude. In particular, the CaHA particles sintered at 970° C. showed a high dose-dependent stimulation efficacy.

The growth factor TGF had a very low influence on resting cells. In case the cell cycle and proliferative competence is however restores by the CaHA particles, TGF even further stimulates cell growth. This shows that the proliferative competence of the cells is fully restored.

### Example 3

### Investigating Prejuvenation and Rejuvenation in an Animal Model

A mouse model is usable as an aging model for prejuvenation and rejuvenation. For this purpose, the mouse model is administered with an injection of D-galactose (e.g., intraperitoneally, i.p. or alternatively subcutaneaously, s.c.). When this is conducted for several weeks (e.g., 8 weeks), the mouse shows symptoms of age. As a control, a comparable mouse is administered with a sham injection such as saline.

To observe an effect of prejuvenation, the mouse is administered with an injection (e.g., subcutaneaously, s.c.) of an injectable composition of the CaHA particles before administration of the D-galactose, i.e., before aging.

To observe an effect of rejuvenation, the mouse is administered with an injection (e.g., subcutaneaously, s.c.) of an injectable composition of the CaHA particles after administration of the D-galactose terminates, i.e., subsequent to aging.

Vascularization and its promotion by the CaHA particles is observable by thermography camera (e.g., FLIR camera).

Improvement of skin barrier by the CaHA particles is observable by detecting the transepidermal water loss (TEWL). A decreased TEWL indicates an increased skin barrier.

Optical coherence tomography (OCT) imaging is usable for detecting an increase of skin thickness by the CaHA particles.

The mouse tissues are investigable histologically. Beneficial effects of CaHA particles are detectable. Increased epidermal and/or dermal thickness is detectable, improved keratosis scoring is detectable, reduced inflammation (hallmark of aging: inflammation) is detectable by nuclei numbering, increases in hair follicles density and numbering are detectable, increases in collagen types I and/or III are detectable, increases in elastin is detectable (e.g., by an Elastin IHC antibody), increases in cells in cell cycle (e.g., by means of detecting KI-67, e.g., proliferation of dermis/epidermis) is detectable, decreases in skin inflammation (e.g., by mouse inflammation panel immunoassay), decreased matrix metalloproteases (MMPs) is detectable (e.g., by a panel immunoassay), decreases in the level of reactive oxygen species (ROS) is detectable, reduced rates of beta-galactosidase are detectable, and/or decreased melanin content is detectable.

The following model equivalents to aging may be used:
Skin thickness decreases (epidermis, dermis): testable in animal model;
Reduction of lipid content and natural water: lower moisture;
Number of Mast cells and Fibroblast decreases: less fibroblast;
Reduction of collagen and elastin content: decrease of hydroxyproline levels and collagen fibers and decrease in elastin;
Collagen bundles become disorganized: collagen fibers are broken, loose, slender and irregular;
Loss of subcutaneous fat: less subcutaneous fat; and
Hair color changes and hair loss: hair color changes and decrease of hair follicles.

1. A method for restoring cell cycle and proliferative competence in resting cells and/or stimulating proliferation of cells, wherein said method comprises the step of contacting the cells with calcium hydroxyapatite particles having a mean particle diameter of from 1 to 500 μm, as determined by light scattering.

2. The method of claim 1, wherein the cells are senescent cells.

3. The method of claim 1, wherein the cells are selected from the group consisting of fibroblasts, fibrocytes, keratinocytes, melanocytes, adipocytes, and skin resident immune cells.

4. The method of claim 1, wherein the calcium hydroxyapatite particles have been sintered at a temperature in the range of from 910 to 1030° C.

5. The method of claim 1, wherein the calcium hydroxyapatite particles are further characterized by at least one of the following features selected from the group consisting of:
(a) the calcium hydroxyapatite particles are spherical or ellipsoid;
(b) the surfaces of the calcium hydroxyapatite particles have pores of an average diameter between 10 and 500 nm at the surface, as determined by Hg-porosimetry;
(c) the calcium hydroxyapatite particles have a mean particle diameter of from 5 to 500 μm, as determined by light scattering;
(d) the calcium hydroxyapatite particles have been sintered at a temperature in the range of from 900 to 1300° C.;
(e) the calcium hydroxyapatite particles have been sintered for 1 to 24 hours; and
(f) a combination of two or more of (a)-(e).

6. The method of claim 1, wherein the calcium hydroxyapatite particles have a mean particle diameter of from 10 to 60 μm, and have been sintered at a temperature in the range of 950 to 980° C. for 3 to 16 hours.

7. The method of claim 1, wherein the cells form part of a tissue.

8. The method of claim 1, wherein the calcium hydroxyapatite particles form part of a composition, comprising:
(A) the calcium hydroxyapatite particles as component A;
(B) one or more cosmetically and/or pharmaceutically acceptable carriers as component B comprising at least one viscous or liquid carrier;
(C) optionally one or more hyaluronic acids as component C;
(D) optionally one or more local anesthetics as component D; and
(E) optionally one or more cosmetically and/or pharmaceutically acceptable additives other than components A, B, C and D as component E.

9. The method of claim 8, wherein the composition is an injectable composition consisting of:
(A) 1 to 80% by weight, referred to dry matter, referred to the injectable composition, of one or more types of calcium hydroxyapatite particles as component A;
(B) 1 to 80% by weight, referred to the injectable composition, of one or more cosmetically and/or pharmaceutically acceptable carriers as component B comprising at least one pasty, viscous or liquid carrier;
(C) 0 to 50% by weight, referred to dry matter, referred to the injectable composition, of one or more hyaluronic acids as component C;
(D) 0 to 10% by weight, referred to the injectable composition, of one or more local anesthetics as component D; and
(E) 0 to 10% by weight, referred to the injectable composition, of one or more cosmetically and/or pharmaceutically acceptable additives other than components A, B, C and D as component E.

10. The method of claim 1, wherein the cells form part of a tissue in a living subject.

11. The method of claim 1, wherein the cells form part of an in vitro cell culture.

12. The method of claim 10, wherein said method is further characterized in that it is a method for a purpose selected from the group consisting of reducing of wrinkles, improving facial lines, breast reconstruction or augmentation, rejuvenation of the skin, buttocks augmentation, remodeling of cheekbones, soft tissue augmentation, filling facial wrinkles, improving glabellar lines, improving nasolabial folds, improving marionette lines, improving buccal commissures, oral commissures, improving peri-lip wrinkles, improving crow's feet, improving subdermal support of the brows, malar and buccal fat pads, improving tear troughs, nose, augmentation of lips, augmentation of cheeks, augmentation of peroral region, augmentation of scars such as acne scars, augmentation of infraorbital region, resolving facial asymmetries, improving jawlines, augmentation of chin, restoring skin tightness, improving evenness of the skin, preventing or restoring discoloration of skin, improving skin glow, restoring skin hydration, improving skin barrier functions, and combinations of two or more thereof.

13. (canceled)

14. A method for treating or preventing a pathologic condition associated with arrested cell cycle, decreased proliferative competence, decreased proliferation rate, or a combination of two or more thereof, or for fostering juvenation of tissue, wherein the method comprises administering to a subject calcium hydroxyapatite particles having a mean particle diameter of from 1 to 500 μm, as determined by light scattering.

15. The method of claim 14, wherein the pathologic condition is age-associated pathologic condition.

16. The method of claim 5, wherein the calcium hydroxyapatite particles are spherical having a D-ratio above 0.7.

17. The method of claim 10, wherein contacting the cells with calcium hydroxyapatite particles is achieved by injecting an injectable composition into the tissue of interest, wherein the injectable composition comprises:
(A) the calcium hydroxyapatite particles as component A;
(B) one or more cosmetically and/or pharmaceutically acceptable carriers as component B comprising at least one viscous or liquid carrier;
(C) optionally one or more hyaluronic acids as component C;
(D) optionally one or more local anesthetics as component D; and
(E) optionally one or more cosmetically and/or pharmaceutically acceptable additives other than components A, B, C and D as component E.

18. The method of claim 11, wherein the cells having restored cell cycle and proliferative competence and/or stimulated to proliferate are introduced into a tissue of interest or the cells having restored cell cycle and proliferative competence and/or stimulated to proliferate are used for producing material of interest.

19. The method of claim 12, wherein said method involves injecting an injectable composition subcutaneously or intradermally.

20. The method of claim 12, wherein said method involves injecting an injectable composition into the connective tissue of the subdermal skin.

21. The method of claim 11, wherein said method is further characterized in that it is a method for a purpose selected from the group consisting of reducing of wrinkles, improving facial lines, breast reconstruction or augmentation, rejuvenation of the skin, buttocks augmentation, remodeling of cheekbones, soft tissue augmentation, filling facial wrinkles, improving glabellar lines, improving nasolabial folds, improving marionette lines, improving buccal commissures, oral commissures, improving peri-lip wrinkles, improving crow's feet, improving subdermal support of the brows, malar and buccal fat pads, improving tear troughs, nose, augmentation of lips, augmentation of cheeks, augmentation of peroral region, augmentation of scars such as acne scars, augmentation of infraorbital region, resolving facial asymmetries, improving jawlines, augmentation of chin, restoring skin tightness, improving evenness of the skin, preventing or restoring discoloration of skin, improving skin glow, restoring skin hydration, improving skin barrier functions, and combinations of two or more thereof.
